(19) United States
(12) Patent Application Publication
(10) Pub. No.: US 2024/0159762 A1
(43) Pub. Date: May 16, 2024

(54) Novel Biomarker

(71) Applicants: Artur Mezheyeuski, Uppsala (SE); Tobias Sjöblom, Knivsta (SE)

(72) Inventors: Artur Mezheyeuski, Uppsala (SE); Tobias Sjöblom, Knivsta (SE)

(21) Appl. No.: 18/282,982

(22) PCT Filed: Mar. 18, 2022
(86) PCT No.: PCT/SE2022/050257

§ 371 Date: Sep. 19, 2023

(30) Foreign Application Priority Data
Mar. 19, 2021 (SE) 2150316-4
Oct. 6, 2021 (SE) 2151223-1

(51) Int. Cl. G01N 33/574

(52) U.S. Cl. CPC G01N 33/57484; G01N 2333/70517; G01N 2333/70596; G01N 2800/52

(57) ABSTRACT

The present invention relates to an in vitro method for the prediction of response to immunotherapy for, or prognosis of survival time of, a subject diagnosed with a cancer, comprising measuring, in a tissue affected by said cancer, a first density D1 of a first cell category consisting of cells positive for CD8 and a second density D2 of a second cell category consisting of cells positive for both CD68 and CD163, determining a relation between D1 and D2; and comparing the determined relation to at least one predetermined reference value predictive of response to immunotherapy, or indicative of a survival time, for said subject.

### FIELD OF THE INVENTION

The present invention relates to the field of detection and analysis of cell populations for purposes of prognosticating disease progression, and in particular for purposes of predicting response to immunotherapy and assessing survival time for cancer patients.

### BACKGROUND

Cancer is a leading cause of death worldwide and it is estimated that about 9.6 million persons died from cancer in 2017. As life expectancies increase due to progress in treatment of other causes of death, the number of cancer cases slowly grows. There is thus a continuous need for novel methods for assessing cancers to inform both patients and caregivers of the status of a patient's individual disease and prospects of future survival.

The TNM classification system of malignant tumors (Brierly et al., 2017) provides internationally agreed standards to describe and categorize cancer stages, published in affiliation with the Union for International Cancer Control (UICC).

An immune scoring system, commercially available under the name Immunoscore® (sometimes abbreviated as “IS” in the present disclosure), which evaluates the abundance of CD3⁺ and CD8⁺ T cells in tumor central regions and at the invasive margin in routinely resected tumors has been proposed (Galon et al., 2006). It has recently been validated as an independent prognostic factor in addition to other clinical parameters, including T and N stage, in colon cancer stage I-III (Pagès et al., 2018). Despite the proven validity of Immunoscore® in colorectal cancer, there is a lack of strong evidence for its prognostic significance in other tumor types.

It has been suggested that CD163⁺ tumor infiltrating macrophages and CD8⁺ cells are crucial prognostic biomarkers in osteosarcoma (Gomez-Brouchet et al., 2017). It was found that the presence of CD68 and CD163 staining were highly correlated together, which was found to suggest that a common subgroup of macrophages may be present. The results were interpreted to demonstrate that high levels of CD163 and CD68 were associated with better overall survival and metastasis-free progression survival. The authors also found that the level of CD8⁺ staining across the patient samples was low with a median staining of 1%. While CD8⁺ cells were detected in more than half of patient samples, their presence was significantly associated with lower rate of metastasis at diagnosis. No relation between the quantified measurements of CD8⁺, CD163⁺ and CD68⁺ cells was studied.

WO2016/134416 discloses a method for providing a prognosis of a subject having diffuse large B-cell lymphoma responding to a treatment regime, the method comprising: determining an immune score for the subject based upon the ratio of a level of any one or more of CD137, CD4, CD8, CD56, TNFα (alpha) and LMO2 in the subject to a level of any one or more of PD-1, PD-L1, CD163, CD68, PD-L2, LAG3, TIM3 and SCYA3(CCL3) in the subject, comparing the immune score to a reference score; wherein the immune score in comparison with the reference score is indicative of the subject's prognosis of responding to the treatment regime. Specific immune scores disclosed in WO2016/134416 all define ratios that include a plurality of marker levels in the numerator and all specified immune scores incorporate PD-1 or PD-L1.

### SUMMARY OF THE INVENTION

The objective of the present invention is to provide alternative and improved biological markers for assessing multiple forms of cancer, and in particular to methods for the prognosis of survival time of a subject diagnosed with cancer.

Thus, in a first aspect, the invention relates to an in vitro method for prediction of response to immunotherapy for, or the prognosis of survival time of, a subject diagnosed with a cancer, comprising
Measuring, in a tissue affected by said cancer, a first density D1 of a first cell category consisting of cells positive for CD8 and a second density D2 of a second cell category consisting of cells positive for at least one of the following: C1q, the combination of CD68 and CD163; and the combination of CD68 and C1q,
determining a relation between D1 and D2; and
comparing the determined relation to at least one predetermined reference value predictive of response to immunotherapy, or indicative of a survival time for said subject.

In one embodiment, the method comprises
obtaining a sample of cancerous tissue from said subject;
Measuring, in said tissue sample, a first density D1 of a first cell category consisting of cells positive for CD8 and a second density D2 of a second cell category consisting of cells positive for at least one of the following: C1q, the combination of CD68 and CD163; and the combination of CD68 and C1q,
determining a relation between D1 and D2;
comparing the determined relation to at least one predetermined reference value indicative of a survival time for said subject; and
determining a prediction of response to immunotherapy or prognosis of survival time for the subject based on the comparison.

In one embodiment, the predetermined reference for prediction of response to immunotherapy for the subject have been determined by
Measuring, in samples of cancerous tissue from each subject in a cohort of subjects diagnosed with said cancer and with a known response to immunotherapy, a first density D1 of a first cell category consisting of cells positive for CD8 and a second density D2 of a second cell category consisting of cells positive for at least one of the following: C1q, the combination of CD68 and CD163; and the combination of CD68 and C1q,
determining a relation between D1 and D2;
comparing the determined relation to the known survival times of the subjects to obtain an association between the relation and the survival time; and
determining the reference values so as to be predictive of response to immunotherapy for a subject diagnosed with the cancer.

In one embodiment, the predetermined reference values for the prognosis of survival time of the subject have been determined by
Measuring, in samples of cancerous tissue from each subject in a cohort of subjects diagnosed with said cancer and with a known survival time, a first density D1 of a first cell category consisting of cells positive for CD8 and a second density D2 of a second cell category consisting of cells positive for at least one of the following: C1q, the combination of CD68 and CD163; and the combination of CD68 and C1q,
determining a relation between D1 and D2;
comparing the determined relation to the known survival times of the subjects to obtain an association between the relation and the survival time; and
determining the reference values so as to be indicative of survival time for a subject diagnosed with the cancer.

In one aspect, the method of measuring relative cell densities in a sample of cancer affected tissue comprising the steps of measuring a first density D1 of a first cell category consisting of cells positive for CD8 in the tissue sample, and a second density D2 of a second cell category consisting of cells positive for at least one of the following: C1q, the combination of CD68 and CD163; and the combination of CD68 and C1q, in the tissue sample, and calculating a relation between D1 and D2.

In some embodiments, the second cell category consists of cells positive for both CD68 and CD163.

In some embodiments, the second cell category consists of cells positive for at least one of C1qA, C1qB, and C1qC, and optionally CD68.

In some embodiments, the determination of the relation between D1 and D2 comprises calculating the ratio D1/(D1+D2) or D1/D2, or an inverse thereof.

In some embodiments, the cancer is selected from colorectal cancer bladder cancer, lung cancer, melanoma, and gastroesophageal adenocarcinoma.

In some embodiments, the calculated ratio is combined with at least one clinical risk factor in determining a prognosis of survival time for the subject.

In some embodiments, the at least one clinical risk factor is selected from the group consisting of subject's sex, microsatellite instability status, tumor sidedness, T stage, N stage, tumor differentiation.

In some embodiments, the measurement of cell densities is performed by analysis of gene expression.

In some embodiments, the measurement of cell densities is performed by counting cells positive for CD8 and cells positive for at least one of the following: C1q, the combination of CD68 and CD163; and the combination of CD68 and C1q in an analysed tissue area, and optionally normalizing against the size of the analysed tissue area.

In some embodiments, the analysed tissue area comprises both tumour centre and invasive margin.

In some embodiments, the counting of cells is facilitated by staining of the tissue with detectable antibodies specific for the CD8, CD68, CD163, C1q, C1qA, C1qB, or C1qC to be detected.

In one aspect, the present invention relates to an in vitro method for the prediction of response to immunotherapy for, or prognosis of survival time of, a subject diagnosed with a cancer, comprising
a) Measuring, in a tissue affected by said cancer, a first concentration C1 of a first group of molecules selected from the group consisting of CD8 and RNA molecules encoding therefore, and a second concentration C2 of a second group of molecules selected from the group consisting of: C1q; the combination of CD68 and CD163; and the combination of CD68 and C1q; and RNA molecules encoding therefore;
b) determining a relation between C1 and C2; and
c) comparing the determined relation to at least one predetermined reference value predictive of response to immunotherapy, or indicative of a survival time, for said subject.

In some embodiments, the the method comprises
obtaining a sample of cancerous tissue from said subject;
performing steps a)-c) of the method according to claim 16; and
determining a prediction of response to immunotherapy or prognosis of survival time for the subject based on the comparison.

In some embodiments, wherein the method is for the prediction of response to immunotherapy for the subject, the predetermined reference values have been determined by
Measuring, in a tissue affected by said cancer, a first concentration C1 of a first group of molecules selected from the group consisting of CD8 and RNA molecules encoding therefore, and a second concentration C2 of a second group of molecules selected from the group consisting of: C1q; the combination of CD68 and CD163; and the combination of CD68 and C1q; and RNA molecules encoding therefore;
determining a relation between C1 and C2; and
comparing the determined relation to the known survival times of the subjects to obtain an association between the relation and the survival time; and
determining the reference values so as to be predictive of response to immunotherapy for a subject diagnosed with the cancer.

In one aspect, the method relates to a method of measuring relative molecule concentrations in a sample of cancer affected tissue comprising the steps of measuring a first concentration C1 of a first group of molecules selected from the group consisting of CD8 and RNA molecules encoding therefore in the tissue sample, and a second concentration C2 of a second group of molecules selected from the group consisting of: C1q; the combination of CD68 and CD163; and the combination of CD68 and C1q; and RNA molecules encoding therefore; in the tissue sample, and calculating a relation between C1 and C2.

In some embodiments, the the second group of molecules consists of CD68 and CD163, or RNA molecules encoding therefore.

In some embodiments, the second group of molecules consists of at least one of C1qA, C1qB, and C1qC, and optionally CD68, or RNA molecules encoding therefore.

In some embodiments, the determination of the relation between C1 and C2 comprises calculating the ratio C1/(C1+C2) or C1/C2, or an inverse thereof.

In some embodiments, the cancer is selected from colorectal cancer, breast cancer, pancreatoduodenal cancer, bladder cancer, lung cancer, melanoma, and gastroesophageal adenocarcinoma.

In some embodiments, the determined relation is combined with at least one clinical risk factor in determining a prognosis of survival time for the subject.

In some embodiments, the at least one clinical risk factor is selected from the group consisting of subject's sex, microsatellite instability status, tumor sidedness, T stage, N stage, tumor differentiation.

In some embodiments, the measurement of concentration is performed by bulk RNA sequencing.

### BRIEF DESCRIPTION OF THE DRAWINGS

FIG. 1: Forest plot of univariate associations of immune cell subclasses, (evaluated as cell densities translated into three-level categorized values,) with OS in therapy-naïve colon cancer patients of stage I-III. Filled squares indicate hazard ratios (HR) and whiskers represent 95% confidence intervals (CI). Cox regression was used for statistical analysis. Asterisks indicate statistically significant associations (p<0.05).

FIG. 2: Kaplan-Meier survival curves in therapy-naïve colon cancer stage I-III patients (n=286). Overall survival (A) and Recurrence-free survival (B) for the patient groups stratified by trichotomized SIA wherein SIA-low was used as reference group. Overall survival (C) and Recurrence-free survival (D) for the patient groups, stratified by IS.

FIG. 3: Predictive accuracy of SIA, IS and clinical parameters for OS (A) and RFS (B) using integrative time-dependent AUC analysis (iAUC) with 1000-fold bootstrap resampling.

FIG. 4: Kaplan-Meier curves and numbers at risk table for OS for patients with colon cancer stage II (A) and metastatic colorectal cancer (B), stratified by trichotomized SIA.

FIG. 5: Overall survival stratified by SIA in six tumor types (Bladder Urothelial Carcinoma (BUC), Gastroesophageal Adenocarcinoma (GA), Lung Carcinoma (LC), Melanoma, Uterine Corpus Endometrial Carcinoma (UCEC) and Ovarian Carcinoma (OC). Patients in BUC, GA, and LC cohorts were stratified in terciles according to SIA level. Melanoma patients were stratified in two groups split by the median. The SIA is prognostic in bladder cancer, cancer of the gastroesophageal junction, lung cancer and melanoma. Overall survival stratified by SIA and analyzed by log-rank did not demonstrate statistically significant associations in UCEC (p value=0.996) and OC (p value=0.383).

FIG. 6: Overall survival stratified by dichotomized ratio between the bulk RNA expression levels of CD8A and each of C1q complement subunits: C1QA, C1QB and C1QC in seven tumor types (three upper panels) and overall survival stratified by dichotomized average bulk RNA expression levels of CD8A and CD3E (IS-like metric). Gene expression and survival data was achieved from the KM plotter database.

FIG. 7: SIA values generated from bulk RNA data by computing the ratio between counts of CD8A and C1QA-C expression in 26 immune checkpoint inhibitor-treated melanomas from patients grouped by response.

### DEFINITIONS

The term “Signature of Immune Activation”, or “SIA”, is used in the present disclosure to denote a biomarker comprising a calculated score based on the relation between the cell densities, in a tissue section of a cancerous tissue, of the entire population of CD8⁺ cells on the one hand and a macrophage subset expressing both CD68 and CD163 on the other.

“OS” is an abbreviation of overall survival.

“RFS” is an abbreviation of recurrence free survival.

The term colon cancer is used to denote a cancer of the colon (classified as anatomical site C18 in the TNM classification) whereas rectal cancer is used to denote a cancer of the rectum (classified as anatomical site C20 in the TNM classification). The term colorectal cancer (or CRC) is used to denote a cancer of the colon or rectum.

The term “gastroesophageal adenocarcinoma” denotes an adenocarcinoma of the oesophagus or gastric region.

C1q, or complement component 1q, is a ˜400 kDa protein complex formed by three subunits each comprising six peptide chains, in total 18 peptide chains. Of these 18 peptide chains, six are A-chains (C1qA), six are B-chains (C1qB) and six are C-chains (C1qC). The term “C1q” refers to, in the context of this disclosure, any one of C1qA, C1qB, and C1qC, as well as the full protein complex, and subunits thereof as well as DNA/RNA encoding such, as given by context. The terms “C1qA”, “C1qB”, and “C1qC” refers to, in the context of this disclosure, the individual peptide chains as well as DNA/RNA encoding such, as given by context.

### DETAILED DESCRIPTION OF THE INVENTION

The present invention builds on the surprising finding that measurement of two specifically defined cell categories in the tumor microenvironment and calculation of their relative densities can be utilized to predict response to immunotherapy and survival of cancer patients. This ratio between cell categories can discriminate responders for immune check-point inhibitor therapy, and also predicted survival better than prior art scoring methods in colon cancer and had the highest relative contribution to survival prediction when compared to established clinical parameters. This ratio was prognostic also in other cancers with high mutation burden, such as those of lung, bladder, esophagus and melanomas.

The predictive and prognostic biomarker according to the present invention confirms the prognostic impact of CD8⁺ cell infiltration and provides a prognostic subset of macrophages that is undetectable using a single-marker approach. Unlike some prior art methods, the present invention does not require independent assessment of the tumor central region and invasive margin. As shown below, the biomarker according to the present invention and the known biomarker Immunoscore® can be used as independent variables in a multivariate analysis. These two metrics are not redundant and presumably capture different aspects of tumor immunity.

Modern in situ analytical techniques, like multimarker immunohistochemistry and multispectral imaging, enable immune cell subclassification into distinct phenotypical and functional groups by multiplex labeling of markers. The present inventors developed two such panels, each consisting of antibodies to five immune markers, for visualization of adaptive and innate immune cells. After cell segmentation of digitized tissue sections as described in the experimental section of the present disclosure, the co-expression pattern of these markers allowed for immune cell sub-classification (Table 1).

TABLE 1
Immune marker combinations in two panels define classes and subclasses of immune cells.

| | immune markers | | | | | | | | | immune | |
|---|---|---|---|---|---|---|---|---|---|---|---|
| CD4 | CD [symbol] | CD [symbol] | FoxP3 | CD [symbol] | CD [symbol] | NKp46 | CD [symbol] | CD [symbol] | CD [symbol] | cell classes | |
| + | | | | | | | | | | panel #1 | CD4 |
| + | − | − | − | + | | | | | | | CD4 + CD45RO+ |
| + | − | − | + | +/− | | | | | | | CD4 Tregs |
| | + | | | | | | | | | | CD8 |
| − | + | − | − | + | | | | | | | CD8 + CD45RO+ |
| − | + | − | + | +/− | | | | | | | CD8 Tregs |
| − | − | + | − | − | | | | | | | [symbol] -cells |
| | | | | + | | | | | | | CD45RO |
| | | | | | − | + | + | − | − | panel #2 | NK |
| | | | | | + | + | + | − | − | | NKT |
| | | | | | | | | + | | | CD68 |
| | | | | | | | | | + | | CD163 |
| | | | | | | | | + | − | | M1-like |
| | | | | | | | | − | + | | CD68 [symbol] CD168+ |
| | | | | | | | | + | + | | M2-like |

[symbol]

indicates data missing or illegible when filed

The major immune cell lineages were defined by single marker expression (CD4, CD8, CD45RO, CD68 and CD163). Further, cells were divided into subclasses according to marker co-expression. Thus, we identified memory CD4 (CD4+CD45RO+) and CD8 (CD8+CD45RO+) lymphocytes, classical T-regulatory (CD4+FoxP3+) and CD8+ Treg (CD8+FoxP3+) cells. As markers of natural killer (NK) cells are less specific, we required co-expression of two markers (CD56 and NKp46) to classify a cell as NK. Similarly, NK T (NKT) cells were defined as those expressing both NK markers and CD3. Finally, the monocyte/macrophage lineage was sub-divided into M1-like macrophages (CD68+CD163−), M2-like macrophages (CD68+CD163+) and CD68-CD163+ cells.

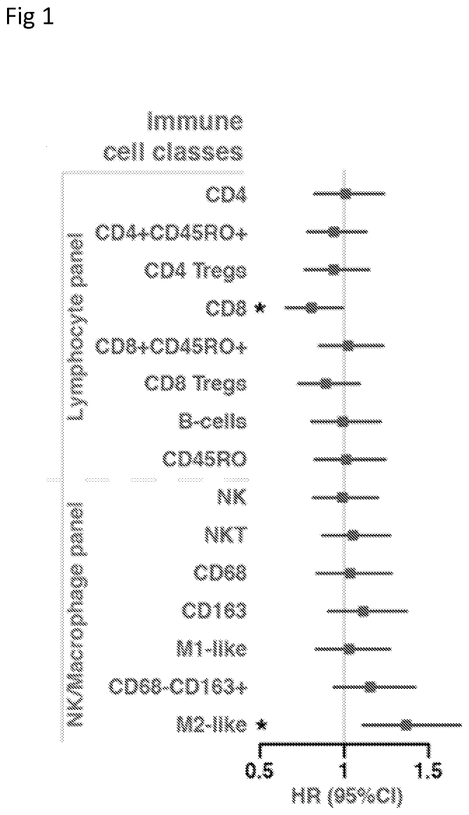

The prognostic impact of the densities of the different immune cells in TNM stage I-III therapy naïve colon cancers (n=286) was evaluated. Only two immune cell classes demonstrated association of cell density with overall survival (OS), namely CD8-positive T lymphocytes (positive association, p=0.042) and M2-like macrophages (negative association, p=0.004) (FIG. 1). Interestingly, positive association with OS was only demonstrated for the entire population of CD8⁺ cells, but not for any of the sub-classified CD8⁺ subsets. Furthermore, neither the pan-macrophage marker CD68 alone nor CD163 alone, which is considered a marker of M2 macrophage differentiation, were associated with survival, whereas a more stringently defined M2-like macrophage subset expressing both CD68 and CD163 was. This M2-like cell subset constituted only 5% of tumor CD68⁺ macrophages and 23% of CD163⁺ cells.

Based on this surprising finding, a signature of immune activation (SIA) based on these two immune cell types was created and forms the basis of parts of the present invention. Without being bound by theory, it is hypothesized that the relative infiltration levels of CD8⁺ cells and M2-like macrophages capture the interplay between anti- and pro-tumoral characteristics of the immune microenvironment.

Furthermore, it was determined that expression of C1q components defines M2-like macrophages in malignant as well as normal tissues. It was also found that a high ratio of CD8 to C1q gene expression is prognostic for overall survival in bladder, esophageal, and rectal carcinomas, and lung adenocarcinoma but not in ovarian and endometrial carcinoma and lung squamous cell carcinoma, largely confirming the results from the immunohistochemistry-based SIA score defined above.

It was also found that the signature of immune activation can discriminate responders for immune check-point inhibitor therapy.

Thus, in a first aspect, the present invention relates to an in vitro method for the prognosis of survival time of a subject diagnosed with a cancer, comprising
Measuring, in a tissue affected by said cancer, a first density D1 of a first cell category consisting of cells positive for CD8 and a second density D2 of a second cell category consisting of cells positive for both CD68 and CD163,
determining a relation between D1 and D2; and
comparing the determined relation to at least one predetermined reference value indicative of a survival time for said subject.

In one embodiment, the method comprises
obtaining a sample of cancerous tissue from said subject;
Measuring, in said tissue sample, a first density D1 of a first cell category consisting of cells positive for CD8 and a second density D2 of a second cell category consisting of cells positive for both CD68 and CD163,
determining a relation between D1 and D2;
comparing the determined relation to at least one predetermined reference value indicative of a survival time for said subject; and
determining a prognosis of survival time for the subject based on the comparison.

In one aspect, the invention relates to a method of measuring relative cell densities in a sample of cancer affected tissue comprising the steps of measuring a first density D1 of a first cell category consisting of cells positive for CD8 in the tissue sample, and a second density D2 of a second cell category consisting of cells positive for both CD68 and CD163 in the tissue sample, and calculating a relation between D1 and D2.

In one aspect, the invention relates to the methods generally as described herein, wherein the second cell category is not defined as cells positive for both CD68 and CD163, but rather defined as cells positive for at least two cell markers selected from the group consisting of CD206, CD200R, CD36, CD204, macrophage activation protein (MAF), and CD86, and the second density D2 is the density of this cell category. In one embodiment the second cell category is defined as cells positive for at least CD206 and CD200R; CD206 and CD36; CD206 and CD204; CD206 and MAF; CD206 and CD86; CD200R and CD36, CD200R and CD204; CD200R and MAF; CD200R and CD86; CD36 and CD204; CD36 and MAF, CD36 and CD86; CD204 and MAF; CD204 and CD86; and/or MAF and CD86.

The sample of tissue affected by the cancer, i.e. the cancerous tissue, may be obtained by surgical resection, biopsy and similar methods, as known in the art.

In one embodiment, the predetermined reference values have been determined by

Measuring, in samples of cancerous tissue from each subject in a cohort of subjects diagnosed with said cancer and with a known survival time, a first density D1 of a first cell category consisting of cells positive for CD8 and a second density D2 of a second cell category consisting of cells positive for both CD68 and CD163,
determining a relation between D1 and D2;
comparing the determined relation to the known survival times of the subjects to obtain an association between the relation and the survival time; and
determining the reference values so as to be indicative of survival time for a subject diagnosed with the cancer.

The relation between D1 and D2 can be calculated in a number of ways, such as a simple ratio between the cell densities (i.e. D1/D2 or D2/D1) or as the relation of one of the cell densities to the sum of cell densities for both cell categories (e.g. D1/(D1+D2) or D2/(D1+D2), or the inverse thereof).

The reference values can be determined in various ways to correlate the relation between D1 and D2 to predicted immunotherapy response for the subject. The reference values may be determined by determining the relation between D1 and D2 in samples from a reference cohort of patients diagnosed with the relevant cancer form, wherein actual immunotherapy response is known for each patient in the reference cohort. Such samples may be obtained from existing collections of tissue samples (e.g. “biobanks”) or new collections of samples collected from specifically selected, diagnosed and/or categorized patients wherein the samples are assessed as being useful in establishing a relevant reference cohort.

The reference values can also be determined in various ways to correlate the relation between D1 and D2 to expected survival time for the subject. The reference values may be determined by determining the relation between D1 and D2 in samples from a reference cohort of patients diagnosed with the relevant cancer form, wherein actual survival time is known for each patient in the reference cohort. Such samples may be obtained from existing collections of tissue samples (e.g. “biobanks”) or new collections of samples collected from specifically selected, diagnosed and/or categorized patients wherein the samples are assessed as being useful in establishing a relevant reference cohort.

In one embodiment, the reference values are determined by obtaining the relation between D1 and D2 for each sample in a reference cohort and transforming the obtained relation to a categorized variable with a set number of levels/categories, such as “high” and “low” (two levels), or “high”, “intermediate” and “low” (three levels) with an essentially equal number of samples in each category.

The reference values may also be obtained by assigning a level of expected survival time for each subject providing a sample to the reference cohort (e.g. “>X weeks” and “≤X weeks” in case of two categories), assigning each obtained relation between D1 and D2 to the relevant survival time category and calculating statistically relevant cut-off value(s) between the categories.

To validate the prognostic value of a biomarker according to the invention with regards to overall survival (OS) and recurrence-free survival (RFS) in colon cancer, a Signature of Immune Activation (“SIA”) was calculated as D1/(D1+D2) for each sample in a colon cancer reference cohort.

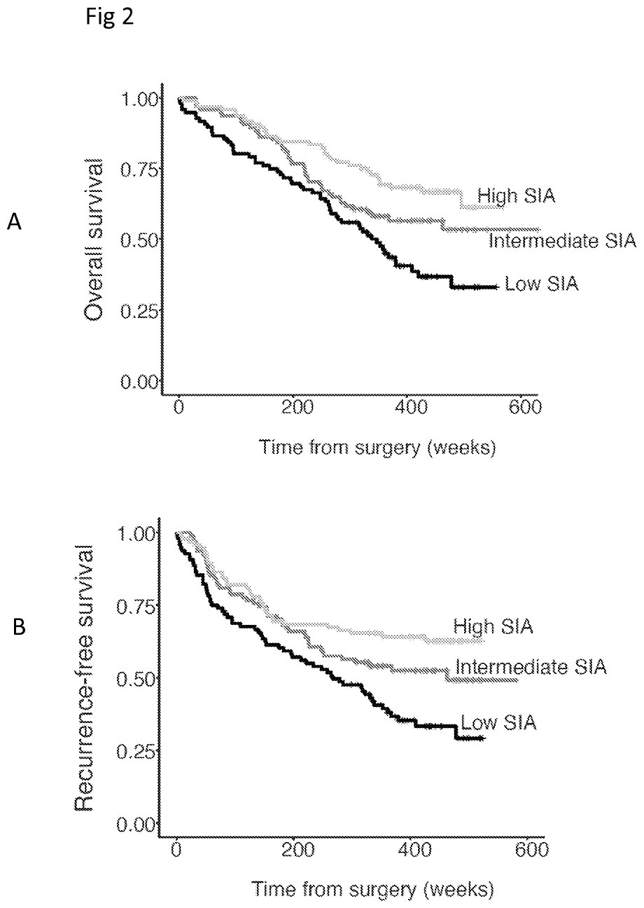

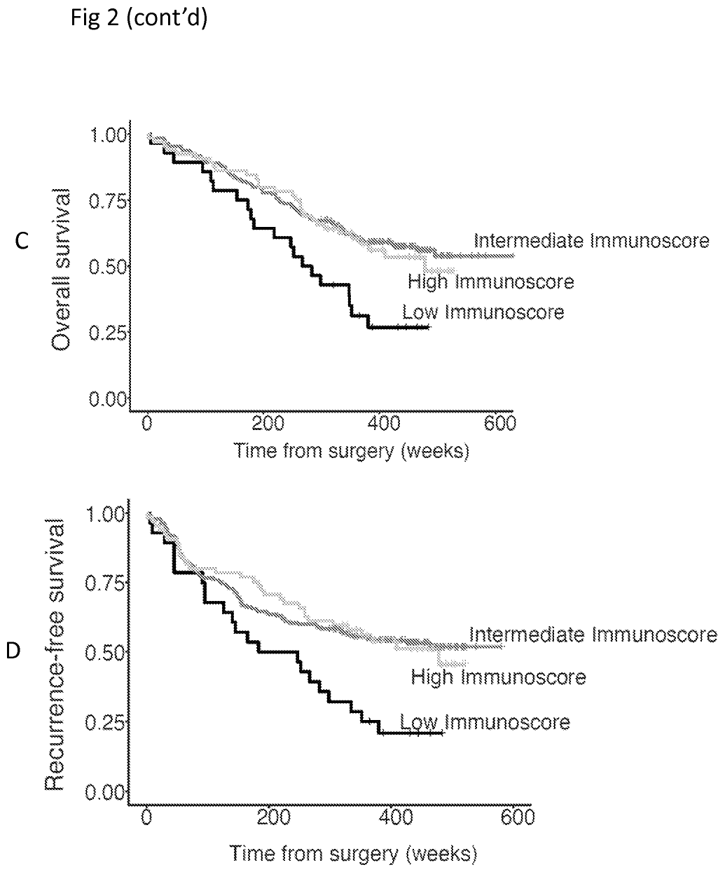

The SIA was then transformed into a three-level categorized variable, i.e. high, intermediate and low, using an unbiased approach with 33.3 and 66.6 percentiles as cutoffs. For comparison, we generated an Immunoscore®-like metric (IS) quantifying densities of CD3⁺ and CD8⁺ cells at the tumor center and invasive margin and translated similarly into a three-grade score (Pagès et al., 2018). IS-low, -intermediate and -high groups were defined as described and IS-low was used as reference group. Both IS and SIA demonstrated strong associations with OS and RFS in colon cancer stage I-III (FIG. 2).

Interestingly, in a multivariable Cox model adjusted for pT stage, pN stage, patient age, gender and MSI status, both SIA and IS were significant independent predictors for OS and RFS (Table 2).

TABLE 2
Univariate and Multivariable analyses of SIA and IS
in therapy-naïve stage I-III colon cancer.

| | Overall survival | | Recurrence-free survival | |
|---|---|---|---|---|
| Co-variable | HR (95% CI) | p value | HR (95% CI) | p value* |
| Unadjusted Cox model, SIA | | | | |
| SIA, three-category | | | | |
| Intermediate vs low | 0.66 (0.44-0.98) | 0.042 | 0.65 (0.44-0.96) | 0.03 |
| High vs low | 0.43 (0.28-0.67) | <0.001 | 0.46 (0.30-0.70) | <0.001 |
| Unadjusted Cox model, IS | | | | |
| IS, three-category | | | | |
| Intermediate vs low | 0.46 (0.28-0.76) | 0.002 | 0.49 (0.30-0.78) | 0.003 |
| High vs low | 0.51 (0.29-0.90) | 0.02 | 0.49 (0.28-0.85) | 0.011 |
| Multivariable Cox model | | | | |
| SIA, three-category | | | | |
| Intermediate vs low | 0.65 (0.42-0.99) | 0.047 | 0.65 (0.43-0.98) | 0.037 |
| High vs low | 0.52 (0.33-0.81) | 0.004 | 0.55 (0.36-0.84) | 0.006 |
| IS, three-category | | | | |
| Intermediate vs low | 0.46 (0.27-0.77) | 0.003 | 0.49 (0.30-0.80) | 0.004 |
| High vs low | 0.62 (0.33-1.16) | 0.137 | 0.62 (0.34-1.12) | 0.114 |
| T stage | | | | |
| T2 vs T1 | 1.31 (0.44-3.95) | 0.626 | 1.12 (0.37-3.36) | 0.84 |
| T3 vs T1 | 1.20 (0.56-2.59) | 0.636 | 1.25 (0.58-2.67) | 0.566 |
| T4 vs T1 | 3.51 (1.52-8.09) | 0.003 | 2.97 (1.30-6.79) | 0.01 |
| N stage | | | | |
| N+ vs N0 | 1.74 (1.18-2.57) | 0.005 | 2.13 (1.47-3.08) | <0.001 |
| Age | | | | |
| Age >75 vs Age =<75 years | 3.49 (2.40-5.09) | <0.001 | 2.60 (1.82-3.72) | <0.001 |
| Sex | | | | |
| Male vs female | 0.85 (0.58-1.23) | 0.38 | 0.90 (0.63-1.28) | 0.562 |
| MSI status | | | | |
| MMR deficient vs proficient | 0.82 (0.50-1.35) | 0.444 | 0.95 (0.60-1.51) | 0.84 |
| MMR missing vs proficient | 1.48 (0.59-3.73) | 0.406 | 1.18 (0.47-2.93) | 0.729 |

MSI: microsatellite instability;
MMR: mismatch repair;
*Wald p value.

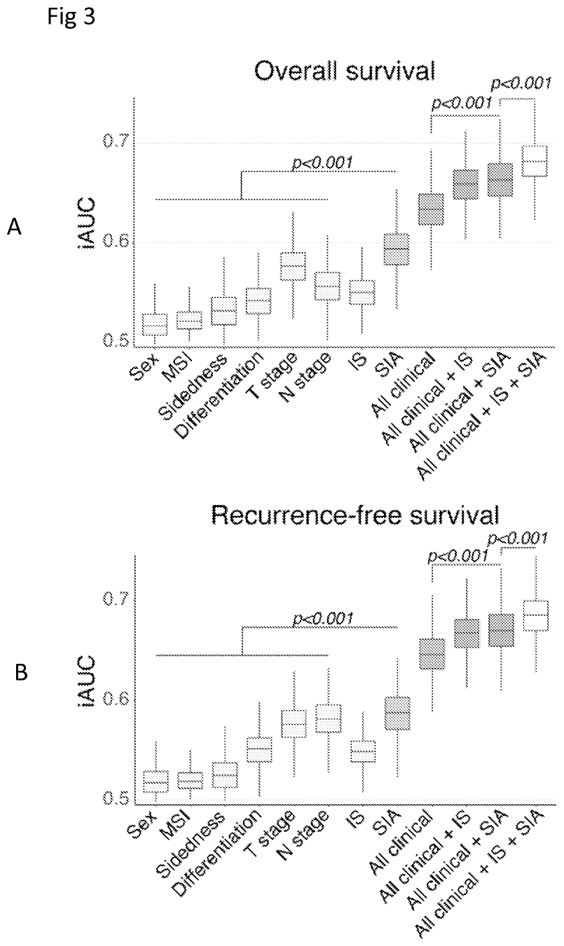

Further, the predictive ability of SIA to IS and well-known clinical risk factors was compared. An integrative time-dependent AUC analysis (iAUC) identified T stage as the strongest clinical predictor for OS (median iAUC 0.58) and N stage for RFS (median iAUC 0.58) (FIG. 3).

Both clinical risk factors and IS were, however, inferior to SIA (median iAUC 0.59 for OS and RFS). Adding SIA to the model with combined clinical parameters further improved the predictive capacity (median iAUC 0.66 and 0.67 for OS and RFS). Finally, when clinical parameters, IS and SIA were integrated in one model, the iAUC was 0.68 and 0.69 for OS and RFS respectively. The relative contribution of SIA to prediction of OS was higher than those of T and N stage (Table 3).

TABLE 3
Relative contribution to the prediction of OS of SIA and clinical
parameters determined using the χ² proportion test.

| | | Relative Chi- | |
|---|---|---|---|
| co-variable | Chi-Square | Square value | p-value |
| SIA | 12.21 | 0.4395 | 0.00223 |
| pT stage | 10.37 | 0.3732 | 0.01567 |
| pN stage | 3.67 | 0.1320 | 0.05549 |
| Differentiation | 0.39 | 0.0140 | 0.82377 |
| Sidedness | 1.09 | 0.0391 | 0.29747 |
| Sex | 0.07 | 0.0024 | 0.79708 |

When IS was included in the model, the relative contribution of SIA and IS exceeded 50% and clearly surpassed the known clinical factors (Table 4).

TABLE 4
Relative contribution to the prediction of OS of SIA, IS-like and
clinical parameters determined using the χ² proportion test.

| | | Relative Chi- | |
|---|---|---|---|
| co-variable | Chi-Square | Square value | p-value |
| SIA | 10.80 | 0.2733 | 0.00452 |
| IS-like | 10.64 | 0.2693 | 0.00488 |
| pT stage | 12.71 | 0.3217 | 0.00530 |
| pN stage | 3.98 | 0.1008 | 0.04599 |
| Differentiation | 0.43 | 0.0109 | 0.80683 |
| Sidedness | 0.95 | 0.0240 | 0.32983 |
| Sex | 0.00059 | 0.000015 | 0.98067 |

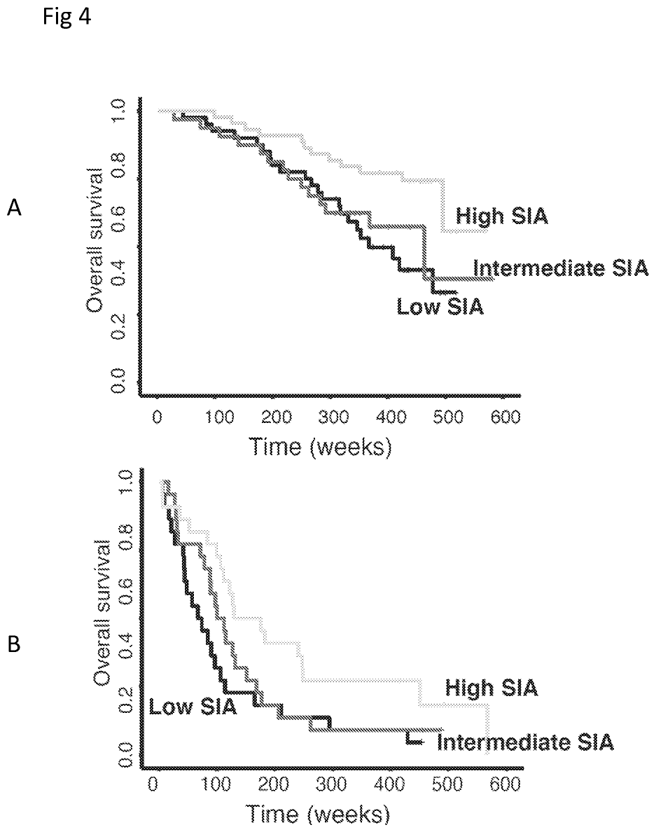

The analysis was repeated for stage II colon cancer patients (n=117) and similar results were observed with SIA stratifying high and low-risk disease (FIG. 4A). Additionally, CRC patients with metastatic disease (n=66) were assessed and stratified into three equally-sized terciles according to SIA. Again, longer OS was seen in the SIA-high group (FIG. 4B)

Thus, SIA demonstrated independent prognostic performance superior to the strongest known clinical predictors (T and N stage), added substantial value to the multivariable prediction model in colon cancer patients of stages I-III, and demonstrated prognostic ability in stage II colon cancer and in metastatic colorectal cancer patients.

It was further investigated whether SIA can be used as a prognostic factor also in other tumor types. Because SIA appears to reflect the balance between anti- and pro-tumoral cell types in the tumor microenvironment, its utility would likely be highest in tumor types with prominent immunogenic properties. We therefore analyzed data obtained from the TCIA project (Charoentong et al., 2017), and ranked different tumors types according to the number of mutations and neoantigens. We then analyzed four independent cohorts of tumors characterized by high mutation and neoantigen count, namely melanoma (n=94) (Stromberg et al., 2009), lung carcinoma (n=251) (Micke et al., 2016), bladder urothelial cancer (n=224) (Hemdan et al., 2014) and gastroesophageal adenocarcinomas (n=121) (Jeremiasen et al., 2020). We also included two cohorts of tumors with lower mutation and neoantigen density, endometrial cancer (n=295) (Huvila et al., 2018) and ovarian cancer (n=141) (Nodin et al., 2010).

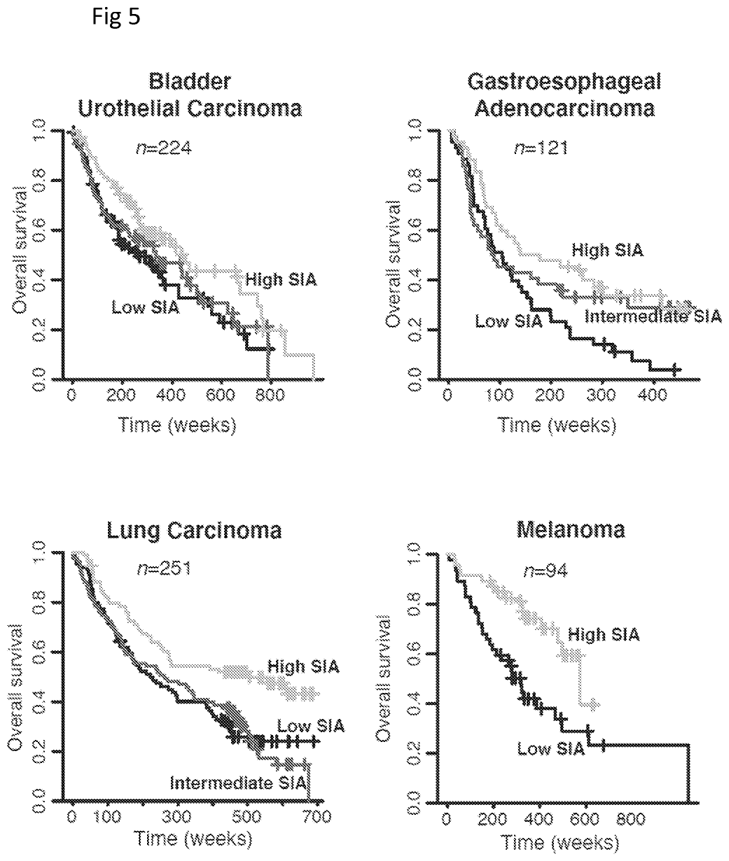

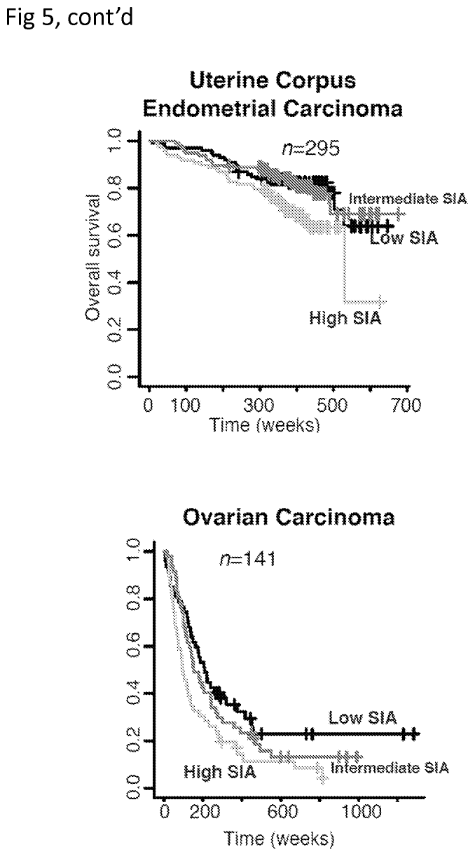

Patients were stratified in tertials according to SIA, with the exception of melanoma where 41% of patients had the highest possible SIA value, and therefore the median was used as cut-off instead. When evaluated in a Cox regression model, high SIA was significantly associated with longer survival in the four tumor types with high mutation and neoantigen count (p values range 0.001-0.037), while no association was seen in endometrial and ovarian cancers (p values 0.996 and 0.399 respectively) (FIG. 5 and Tables 5-8).

Relative hazards, estimated in univariate Cox proportional hazards model, using Overall survival as the end points are shown in Tables 3-6.

TABLE 5
Bladder Urothelial Carcinoma

| Co-variable | HR (95% CI) | p value |
|---|---|---|
| SIA, three-category | | |
| High vs low | 0.64 (0.42-0.85) | p = 0.038 |
| Intermediate vs low | 0.88 (0.67-1.09) | p = 0.548 |

TABLE 6
Gastroesophageal Adenocarcinoma

| Co-variable | HR (95% CI) | p value |
|---|---|---|
| SIA, three-category | | |
| High vs low | 0.53 (0.29-0.78) | p = 0.011 |
| Intermediate vs low | 0.69 (0.44-0.93) | p = 0.128 |

TABLE 7
Lung Carcinoma

| Co-variable | HR (95% CI) | p value |
|---|---|---|
| SIA, three-category | | |
| High vs low | 0.54 (0.34-0.74) | p = 0.002 |
| Intermediate vs low | 1.02 (0.84-1.19) | p = 0.931 |

TABLE 8
Melanoma

| Co-variable | HR (95% CI) | p value |
|---|---|---|
| SIA, two-category | | |
| High vs low | 0.39 (0.075-0.71) | p = 0.003 |

No association was seen in endometrial and ovarian cancers (p values 0.996 and 0.399 respectively).

Further, according to the iAUC analysis in the four cohorts, SIA surpassed IS for prediction of OS, demonstrating median iAUC ranging from 0.55 in bladder cancer to 0.61 in melanoma (Table 9). Interestingly, the time-dependent discrimination properties of SIA in colon cancer were higher than the recently published validated performance of IS (iAUC 0.57 (Pagès et al., 2018)).

TABLE 9
Predictive accuracy of SIA and IS for OS in four cancer cohorts using integrative
time-dependent AUC analysis (iAUC) with 1000-fold bootstrap resampling.

| | | Summary of time-dependent AUC bootstrap resampling | | | | | |
|---|---|---|---|---|---|---|---|
| cancer | metric | Min. | 1st Qu. | Median | Mean | 3rd Qu. | Max. |
| Bladder | IS-like | 0.4903 | 0.5067 | 0.5142 | 0.5158 | 0.5225 | 0.5660 |
| | SIA | 0.4954 | 0.5339 | 0.5507 | 0.5504 | 0.5656 | 0.6322 |
| Gastroesoph | IS-like | 0.4986 | 0.5163 | 0.5312 | 0.5328 | 0.5473 | 0.6011 |
| | SIA | 0.5006 | 0.5560 | 0.5712 | 0.5721 | 0.5898 | 0.6526 |
| Lung | IS-like | 0.5006 | 0.5410 | 0.5543 | 0.5542 | 0.5679 | 0.6079 |
| | SIA | 0.5011 | 0.5502 | 0.5677 | 0.5660 | 0.5809 | 0.6302 |
| Melanoma | IS-like | 0.4866 | 0.5177 | 0.5374 | 0.5407 | 0.5594 | 0.6500 |
| | SIA | 0.4969 | 0.5876 | 0.6142 | 0.6133 | 0.6404 | 0.7373 |

The SIA is thus a prognostic factor in multiple cancer tumor types.

Consequently, in one embodiment of the invention, the cancer is a cancer having a median number of mutations and/or neoantigens above 100. Median numbers of mutations and neoantigens may be obtained from The Cancer Immunome Atlas (TCIA) project (tcia.at/home) (Charoentong et al., 2017)

Single-cell RNA sequencing data from 9 colorectal tumors (Lee et al., 2020) was analyzed. 6520 macrophages of the three subclasses defined by CD68 and CD163 gene expression were identified, comprising 17% of M1-like macrophages, 79% of M2-like macrophages and 4% of CD68-CD163+ cells. Analysis of differentially expressed genes in these macrophages demonstrated that cells of the M2-like subgroup overexpressed C1QA, C1QB, and C1QC, together encoding C1q, a subcomponent of the C1 complement complex. This was observed also in two additional datasets, where C1QA-C were among the top upregulated genes in M2-like macrophages in lung cancer (n=13286 cells) (Lambrechts et al., 2018) and uveal melanoma (n=25413 cells) (Durante et al., 2020). In CRC and lung cancer it was also observed high expression of APOE, encoding Apolipoprotein E, restricted to M2-like macrophages. However, this was not the case in uveal melanoma, where APOE was highly expressed also in M1-like macrophages. By analysis of the complete datasets of three single-cell collections from cancer tissue (54259 cells in CRC, 32439 in lung cancer and 97550 in uveal melanoma), it was observed that C1QA, C1QB, and C1QC were expressed almost exclusively in macrophages whereas APOE was expressed also in other cell types , which is in line with previous findings.

It was further investigated if the transcription profile of macrophages differs in tumors, adjacent tissues and in non-diseased organs. First, macrophages were compared from tumor and peritumoral tissues in CRC and lung cancer and found the same level of expression of C1QA-C and APOE in macrophages from both locations. Next, scRNAseq data from 15 different non-malignant organs of the same individual were explored (He et al., 2020) to determine if C1QA-C and APOE-producing cells are present also in normal organs. Only a small fraction of cells expressed C1QA-C (average 4% across all organs, ranging from 0.12 in lymph node to 17-19% in liver), whereas a higher fraction expressed APOE (average 17%, from 0% in blood to 64% in skin). The majority of C1QA-C expressing cells were macrophages (defined by CD68 and/or CD163 positivity) ranging from 45-56% of the positive cells in lymph node to 91-93% in liver.

When analyzing macrophage subclasses, C1QA-C expression was characteristic for M2-like macrophages but very low in M1-like cells, while APOE expression in macrophages was lower and lacked association with differentiation. Taken together, the expression of C1q components defines M2-like macrophages in malignant as well as normal tissues.

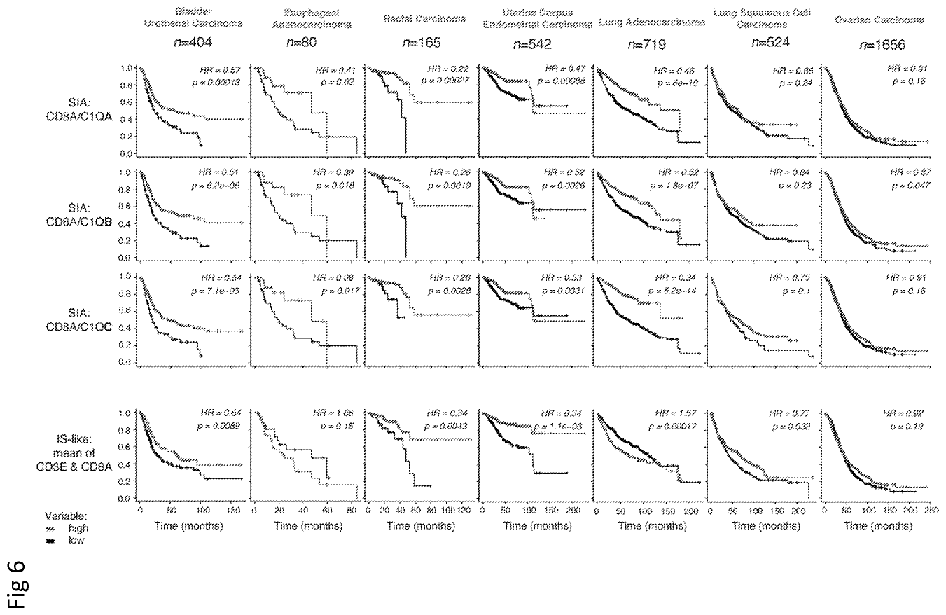

As the C1QA-C expression in cancers was mainly detected in M2-like macrophages, the synthesis of complement C1q components analyzed at the bulk RNA level is, in one embodiment of the invention, used to define the second cell category according to the invention. Bulk RNA expression data was extracted from the KM plotter database (Nagy et al., 2021), the ratio between the expression level of CD8A and either C1QA, C1QB or C1QC was dichotomized, and survival analysis for bladder, esophageal, rectal, endometrial and ovarian carcinomas lung adenocarcinoma and lung squamous cell carcinoma was performed. A high ratio was associated with improved survival in all analyzed tumor types except lung squamous cell carcinoma and ovarian cancer (FIG. 6), largely confirming the results from the immunohistochemistry based SIA score (cf. FIG. 5). Importantly, an Immunoscore-like metric generated from bulk RNA datasets had inferior performance, with the exception of endometrial carcinoma (FIG. 6). Thus, the ratio between CD8A and either C1QA, C1QB or C1QC in bulk tumor gene expression data is prognostic in at least five tumor types.

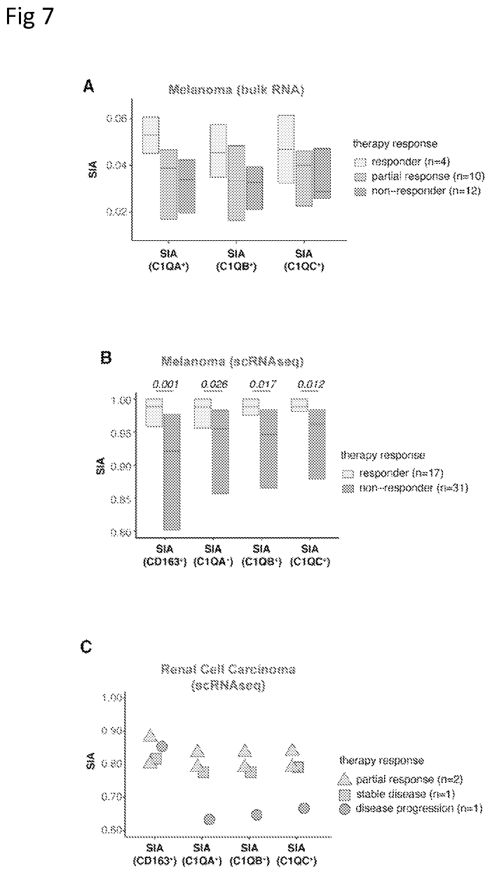

Finally, it was investigated if SIA can discriminate responders for immune check-point inhibitor therapy. Bulk RNA from melanomas in patients treated with anti-PD-1 therapy (Hugo et al., 2016) were analyzed. Then, the ratio between CD8A and either C1QA, C1QB or C1QC gene expression was computed. Interestingly, the responders (n=4) had higher SIA values compared to partial responders (n=10) and non-responders (n=12) (FIG. 7A).

To enable more accurate signature estimation, also single-cell sequencing data from melanoma patients treated with anti-PD1 and/or anti-CTLA4 (n=48) (Sade Feldman et al., 2018) were analyzed and SIA was computed using single cell gene expression levels of CD8A to define CD8+ cells and a combination of the expression of CD68 and either of CD163, C1QA, C1QB or C1QC to define M2-like macrophages. A clear association between high SIA scores and response to immune check-point inhibitor therapy was observed with SIA derived from CD8A and CD68+CD163 (p=0.001), CD68+C1QA (p=0.026), CD68+C1QB (p=0.017) or CD68+C1QC (p=0.012), respectively (FIG. 7B).

To verify the accuracy of SIA for the prediction of treatment response a ROC analysis was performed, which yielded the area under curve (AUC) ranging from 0.70 (for SIA derived from CD8A and CD68+C1QA) to 0.79 (for SIA derived from CD8A and CD68+CD163). Single-cell RNA sequencing data from renal cell carcinomas (Bi et al., 2021) was analyzed, of which four patients received immune checkpoint therapy and had objective response records. Two patients with partial response had higher SIA, derived from cell counts considering complement co-expression by M2-like macrophages, in comparison to one patient with tumor progression (FIG. 7C). Taken together, these observations support that SIA can predict response to treatment with immune checkpoint inhibitors in melanoma and other tumor types.

In one embodiment, the cancer is selected from colon cancer, colorectal cancer, bladder cancer, lung cancer, melanoma, and gastroesophageal adenocarcinoma.

In one embodiment, the determined relation is combined with at least one clinical risk factor in determining a prognosis of survival time for the subject.

In one embodiment, the at least one clinical risk factor is selected from the group consisting of subject's sex, microsatellite instability status, tumor sidedness, T stage, N stage, tumor differentiation.

In embodiments of the invention according to the above aspects, the measurement of cell densities is performed by counting cells positive for CD8 and cells positive for both CD68 and CD163 in an analysed tissue area, and optionally normalizing against the size of the analysed tissue area.

In embodiments of the invention according to the above aspects, the analysed tissue area comprises both tumour centre and invasive margin.

In embodiments of the invention according to the above aspects, the counting of cells is facilitated by immunofluorescence staining of the tissue with detectable antibodies specific for the applicable cell markers (e.g. CD8, CD68, and CD163). The counting of cells may generally be done by allowing detectable compounds capable of specific affinity binding (commonly known as “affinity binders”) to the applicable cell markers to bind to cells in a tissue section of a tissue of interest, detecting the quantity of bound detectable compound and correlating the detected quantity to the size of the tissue section or correlating the detected quantity of each cell marker to the total quantity of all, or a subset, of the cell markers. Affinity binders include antibodies, both monoclonal and polyclonal, and antibody fragments comprising at least the variable regions of both heavy and light immunoglobulin chains held together (usually by disulfide bonds) so as to preserve the antibody-binding site. Types of antibody fragments include Fab, Fab′, F(ab′)2, Fv, rlgG, single chain variable fragments (scFv), scFV dimers (diabodies), scFV fusion proteins (e.g. scFV-Fc), affibodies etc. Other types of affinity binders such as molecularly imprinted polymers may also be utilized. Detectable compounds are also known in the art and comprise e.g. fluorescent moieties, metals (e.g. gold nanoparticles), and moieties that may be used to bind further detectable compounds, e.g. streptavidin or biotin.

In one aspect, the invention relates to a kit of parts comprising a set of reagents adapted to facilitate counting of cells positive for CD8 and cells positive for both CD68 and CD163, or other applicable markers defining a cell category of interest as disclosed herein. Such reagents may be selected from the reagents listed in Table 12 and reagents with equivalent functionality in detection of cells expressing the cell markers of interest.

The following examples are included to further illustrate and facilitate understanding of the invention. They are not to be construed as limiting the scope of the invention, which is that of the appended claims. All references cited herein are expressly incorporate by reference in their entirety.

### EXAMPLES

### Materials and Methods

### Study Cohorts and Tissue Microarrays

The colorectal cancer (CRC) cohort consists of prospectively collected CRC patients living in Uppsala County, Sweden, most of whom have been included in the Uppsala-Umeå Comprehensive Cancer Consortium (U-CAN, u-can.uu.se). In total, 937 patients were diagnosed with CRC between 2010 and 2014 in the Uppsala region. Of them, 746 (80%) were included in a TMA. For the present study, only patients with TMA material from primary tumors were selected. After the staining procedures and quality control, 497 patients had data from both immune panels of whom 286 patients had TNM I-III stage therapy naïve colon cancer. The clinicopathological characteristics of the included patients and their tumors are presented in Table 10.

All patients received stage-stratified standard of care according to the Swedish national guidelines from 2008. According to the guidelines, colon tumors were recommended primary surgery and adjuvant chemotherapy if risk factors for recurrence were present. If the colon tumor was considered inextirpable/borderline resectable, neoadjuvant chemotherapy was administered to shrink the tumor before surgery. Rectal cancers received preoperative or neo-adjuvant radiotherapy/chemoradiotherapy stratified according to risk for locoregional or systemic recurrence. Formalin-fixed paraffin-embedded tissue blocks of primary tumors and distant metastases were used to construct TMAs. Each case was represented on the TMA with cores derived from the central part of the tumor and from the invasive margin. The study was performed within ethical permits from the regional ethical committee in Uppsala, Sweden.

TABLE 10
Baseline clinicopathological characteristics in the colorectal cancer cohort.
Patient data shown in subgroups with successful staining available from each
of the two multiplex panels (left column. TIL panel and middle column. NK/MF
panel) and in a subgroup where both panels were available (right column.
SIA panel). Values are shown as the number of cases (percentage) unless indicated
otherwise. Percentages may not add to 100% due to rounding.

| | TIL Panel | | NK/MF Panel | | SIA group | |
|---|---|---|---|---|---|---|
| Characteristics | (n = 520) | | (n = 508) | | (n = 497) | |
| Age | | | | | | |
| Average age in years ± SD | 69.83 ± 12.02 | | 69.76 ± 11.89 | | 69.86 ± 11.86 | |
| <75 and =75 years old | 338 | (65%) | 331 | (65.2%) | 323 | (65%) |
| >75 years old | 182 | (35%) | 117 | (34.8%) | 174 | (35%) |
| Gender | | | | | | |
| Male | 282 | (54.2%) | 274 | (53.9%) | 268 | (53.9%) |
| Female | 238 | (45.8%) | 234 | (46.1%) | 229 | (46.1%) |
| Location | | | | | | |
| Colon | 365 | (70.2%) | 355 | (69.9%) | 351 | (70.6%) |
| Rectum | 155 | (29.8%) | 153 | (30.1%) | 146 | (29.4%) |
| T Stage | | | | | | |
| 0 | 3 | (0.6%) | 5 | (1%) | 2 | (4%) |
| 1 | 39 | (7.5%) | 37 | (7.3%) | 37 | (7.4%) |
| 2 | 61 | (11.7%) | 60 | (11.8%) | 58 | (11.7%) |
| 3 | 309 | (59.4%) | 304 | (59.8%) | 300 | (60.4%) |
| 4 | 99 | (19%) | 95 | (18.7%) | 93 | (18.7%) |
| Missing data | 9 | (1.7%) | 7 | (1.4%) | 7 | (1.4%) |
| N Stage | | | | | | |
| 0 | 273 | (52.5%) | 266 | (52.4%) | 261 | (52.5%) |
| 1 | 237 | (45.6%) | 236 | (46.5%) | 230 | (46.3%) |
| Missing data | 10 | (1.9%) | 6 | (1.2%) | 6 | (1.2%) |
| M Stage | | | | | | |
| 0 | 437 | (84%) | 426 | (83.9%) | 418 | (84.1%) |
| 1 | 77 | (14.8%) | 77 | (15.2%) | 74 | (14.9%) |
| Missing data | 6 | (1.2%) | 5 | (1%) | 5 | (1%) |
| pTNM stage | | | | | | |
| 0 | 4 | (0.8%) | 5 | (1%) | 3 | (0.6%) |
| 1 | 85 | (16.3%) | 82 | (16.1%) | 80 | (16.1%) |
| 2 | 172 | (33.1%) | 166 | (34.6%) | 165 | (33.2%) |
| 3 | 179 | (34.4%) | 176 | (34.6%) | 173 | (34.8%) |
| 4 | 77 | (14.8%) | 77 | (15.2%) | 74 | (99.6%) |
| Missing data | 3 | (0.6%) | 2 | (0.4%) | 2 | (0.4%) |
| Differentiation Grade | | | | | | |
| Low | 368 | (70.8%) | 356 | (70.1%) | 353 | (71%) |
| High | 84 | (16.2%) | 83 | (16.3%) | 81 | (16.3%) |
| Missing data | 68 | (13.1%) | 69 | (13.6%) | 63 | (12.7%) |
| Neural Invasion | | | | | | |
| No | 361 | (69.4%) | 352 | (69.3%) | 343 | (69%) |
| Yes | 80 | (15.4%) | 78 | (15.4%) | 78 | (15.7%) |
| Missing data | 79 | (15.2%) | 78 | (15.4%) | 76 | (15.3%) |
| Vascular Invasion | | | | | | |
| No | 132 | (25.4%) | 326 | (64.2%) | 317 | (63.8%) |
| Yes | 331 | (63.7%) | 125 | (24.6%) | 125 | (25.2%) |
| Missing data | 57 | (11%) | 57 | (11.2%) | 55 | (11.1%) |
| MSI/MMR Status | | | | | | |
| MMR proficient | 441 | (84.8%) | 432 | (85%) | 424 | (85.3%) |
| MMR deficient | 66 | (12.7%) | 64 | (12.6%) | 62 | (12.5%) |
| Missing data | 13 | (2.5%) | 12 | (2.4%) | 11 | (2.2%) |
| BRAF Mutation | | | | | | |
| No | 206 | (39.6%) | 201 | (39.6%) | 197 | (39.6%) |
| Yes | 46 | (8.8%) | 45 | (8.9%) | 45 | (9.1%) |
| Missing data | 268 | (51.5%) | 262 | (51.5%) | 255 | (51.3%) |
| Neoadjugant Treatment | | | | | | |
| (in Rectal only) | | | | | | |
| No | 51 | (32.9%) | 48 | (31.4%) | 47 | (32.3%) |
| Yes | 91 | (58.7%) | 93 | (60.8%) | 87 | (59.6%) |
| Missing data | 13 | (8.4%) | 12 | (7.8%) | 12 | (8.2%) |
| Neoadjugant Treatment | | | | | | |
| Type (in Rectal only) | | | | | | |
| scRT + delay | 32 | (20.6%) | 31 | (20.3%) | 31 | (21.2%) |
| scRT + immediate | 29 | (18.7%) | 28 | (18.3%) | 28 | (19.2%) |
| scRT + CT | 10 | (6.5%) | 11 | (7.2%) | 9 | (6.2%) |
| CRT | 8 | (5.2%) | 10 | (6.5%) | 8 | (5.5%) |
| CT | 1 | (0.6%) | 1 | (0.7%) | 0 | |
| lcRT | 1 | (0.6%) | 1 | (0.7%) | 1 | (0.7%) |
| Missing data | 22 | (14.2%) | 22 | (14.2%) | 21 | (14.4%) |

(MSI—microsatellite instability; MMR—mismatch repair; RT—radiotherapy; scRT—short-course radiotherapy; lcRT—long-course radiotherapy CT—chemo therapy; CRT—chemo-radio therapy)

The melanoma cohort encompassed TMA cores from 94 patients diagnosed with primary cutaneous malignant melanoma in the Uppsala region, Sweden, from 1980 to 2004 (Stromberg et al., 2009). The study was approved by the research ethics committee at Uppsala University, Uppsala, Sweden.

The lung cancer cohort encompassed TMA cores from 251 patients diagnosed with Non-Small Cell Lung Cancer who underwent surgical treatment at Uppsala University Hospital, Sweden from 2006 to 2010 (Micke et al., 2016). The study was performed under a permit from the regional ethical committee in Uppsala.

The gastroesophageal cancer cohort included TMA cores from 121 patients with chemoradiotherapy-naïve gastroesophageal adenocarcinomas who underwent surgery at the University Hospitals of Lund and Malmö from 2006 to 2010 (Jeremiasen et al., 2020). The study was performed under a permit from the regional ethical committee in Lund.

The urothelial cancer cohort encompassed TMA cores collected from primary urothelial tumors from 224 patients undergoing surgery at Uppsala University Hospital between 1984 and 2005 (Hemdan et al., 2014). The study was performed under a permit from the regional ethical committee in Uppsala.

The uterine corpus endometrial carcinoma cohort consisted of TMA cores from 295 uterine carcinomas from patients surgically treated at Turku University Hospital, Finland, between 2004-2007 (Huvila et al., 2018). The study was performed under a permit from the ethical review board in Helsinki.

The ovarian carcinoma cohort was presented as TMA cores from invasive ovarian cancer cases, derived from two pooled prospective, population-based cohorts; the Malmö Diet and Cancer Study and the Malmö Preventive Project (Nodin et al., 2010). The study was performed under a permit from the regional ethical committee in Lund.

The clinicopathological characteristics of the included patients in the melanoma, lung cancer, gastroesophageal cancer, urothelial cancer, UCEC and ovarian carcinoma cohorts and their tumors are presented in Table 11

TABLE 11
Baseline clinicopathological characteristics of validation cohorts. Patient data shown
in groups where SIA data was available. Values are shown as the number of cases (percentage)
unless indicated otherwise. Percentages may not add to 100% due to rounding.

| | | | Non Small | | Urine | | | | | | | |
|---|---|---|---|---|---|---|---|---|---|---|---|---|
| | Endometrial | | Cell Lung | | Bladder | | Ovarian | | Gastroesophageal | | | |
| | Cancer | | Cancer | | Cancer | | Cancer | | Cancer | | Melanoma | |
| Patient | | | | | | | | | | | | |
| sample size | | | | | | | | | | | | |
| and median | | | | | | | | | | | | |
| survival | | | | | | | | | | | | |
| time | | | | | | | | | | | | |
| Total | 295 | (26.1%) | 251 | (22.2%) | 224 | (19.8%) | 141 | (12.5%) | 127 | (11.2%) | 94 | (8.3%) |
| number of | | | | | | | | | | | | |
| patients (%) | | | | | | | | | | | | |
| Median | 545.08 ± 14.19^{b} | | 276 ± 43.58 | | 348 ± 50.85 | | 151 ± 18.55 | | 113 ± 20.80 | | 478.4 ± 56.96 | |
| survival | | | | | | | | | | | | |
| time^{a} ± SD | | | | | | | | | | | | |
| (weeks) | | | | | | | | | | | | |
| Age at | | | | | | | | | | | | |
| diagnosis | | | | | | | | | | | | |
| Mean ± SD | 66 ± 10.47 | | 67.19 ± 7.45 | | 70.69 ± 11.81 | | 63.70 ± 8.39 | | 70.8 ± 11.04 | | 60.45 ± 14.61 | |
| Median ± SD | 66 ± 10.47 | | 67 ± 7.45 | | 72 ± 11.81 | | 63 ± 8.39 | | 72 ± 11.04 | | 61.09 ± 14.61 | |
| ≤Median | 152 | (51.5%) | 132 | (52.6%) | 115 | (51.3%) | 73 | (51.8%) | 64 | (50.4%) | 47 | (50%) |
| >Median | 143 | (48.5%) | 119 | (47.4%) | 109 | (48.7%) | 68 | (48.2%) | 63 | (49.6%) | 47 | (50%) |
| Sex | | | | | | | | | | | | |
| Female | 295 | (100%) | 128 | (51%) | 50 | (22.3%) | 141 | (100%) | 29 | (22.8%) | 44 | (46.8%) |
| Male | — | | 123 | (49%) | 174 | (77.7%) | — | | 98 | (77.2%) | 50 | (53.2%) |
| pT | | | | | | | | | | | | |
| classification | | | | | | | | | | | | |
| pT0 | 0 | | 0 | | 102 | (45.5%) | 0 | | 0 | | 0 | |
| pT1 | 0 | | 113 | (45%) | 95 | (42.4%) | 0 | | 7 | (5.5%) | 23 | (24.5%) |
| pT2 | 0 | | 102 | (40.6%) | 18 | (8%) | 0 | | 22 | (17.3%) | 27 | (28.7%) |
| pT3 | 0 | | 28 | (11.2%) | 7 | (3.1%) | 0 | | 77 | (60.6%) | 23 | (24.5%) |
| pT4 | 0 | | 8 | (3.2%) | 2 | (0.9%) | 0 | | 21 | (16.5%) | 15 | (16%) |
| Missing data | 295 | (100%) | 0 | | 0 | | 141 | (100%) | 0 | | 6 | (6.4%) |
| pN | | | | | | | | | | | | |
| classification | | | | | | | | | | | | |
| pN0 | 0 | | 193 | (76.9%) | 24 | (10.7%) | 0 | | 36 | (28.3%) | 0 | |
| pN1 | 0 | | 29 | (11.6%) | 5 | (2.2%) | 0 | | 26 | (20.5%) | 0 | |
| pN2 | 0 | | 29 | (11.6%) | 0 | | 0 | | 33 | (26%) | 0 | |
| pN3 | 0 | | 0 | | 0 | | 0 | | 32 | (25.2%) | 0 | |
| Missing data | 295 | (100%) | 0 | | 195 | (87.1%) | 141 | (100%) | 0 | | 94 | (100%) |
| pM | | | | | | | | | | | | |
| classification | | | | | | | | | | | | |
| pM0 | 0 | | 0 | | 60 | (26.8%) | 0 | | 116 | (91.3%) | 0 | |
| pM1 | 0 | | 0 | | 18 | (8%) | 0 | | 3 | (2.4%) | 0 | |
| pM2 | 0 | | 0 | | 0 | | 0 | | 8 | (6.3%) | 0 | |
| Missing data | 295 | (100%) | 251 | (100%) | 146 | (65.2%) | 141 | (100%) | 0 | | 94 | (100%) |
| Clinical | | | | | | | | | | | | |
| stage at | | | | | | | | | | | | |
| diagnosis | | | | | | | | | | | | |
| 1 | 242 | (82%) | 155 | (61.8%) | 0 | | 23 | (16.3%) | 0 | | 0 | |
| 2 | 8 | (2.7%) | 53 | (21.1%) | 0 | | 16 | (11.3%) | 0 | | 0 | |
| 3 | 39 | (13.2%) | 36 | (14.3%) | 0 | | 72 | (51.1%) | 0 | | 0 | |
| 4 | 6 | (2%) | 7 | (2.8%) | 0 | | 19 | (13.5%) | 0 | | 0 | |
| Missing data | 0 | | 0 | | 224 | (100%) | 11 | (7.8%) | 127 | (100%) | 94 | (100%) |
| Differentiation | | | | | | | | | | | | |
| grade/ | | | | | | | | | | | | |
| Histological | | | | | | | | | | | | |
| differentiation | | | | | | | | | | | | |
| G1 Well | 247 | (83.7%) | 0 | | 71 | (31.7%) | 4 | (2.8%) | 77 | (60.6%) | 0 | |
| differentiated | | | | | | | | | | | | |
| (Low grade) | | | | | | | | | | | | |
| G2 | — | | — | | — | | 31 | (22%) | — | | — | |
| Moderately | | | | | | | | | | | | |
| differentiated | | | | | | | | | | | | |
| (Intermediate | | | | | | | | | | | | |
| grade) | | | | | | | | | | | | |
| G3 Poorly | 48 | (16.3%) | 0 | | 153 | (68.3%) | 106 | (75.2%) | 50 | (39.4%) | 0 | |
| differentiated | | | | | | | | | | | | |
| (High grade) | | | | | | | | | | | | |
| Missing data | 0 | | 251 | (100%) | 0 | | 0 | | 0 | | 94 | (100%) |
| WHO | | | | | | | | | | | | |
| performance | | | | | | | | | | | | |
| status | | | | | | | | | | | | |
| 0 | 0 | | 153 | (61%) | 0 | | 0 | | 0 | | 0 | |
| 1 | 0 | | 95 | (37.8%) | 0 | | 0 | | 0 | | 0 | |
| 2 | 0 | | 3 | (1.2%) | 0 | | 0 | | 0 | | 0 | |
| ≥3 | 0 | | 0 | | 0 | | 0 | | 0 | | 0 | |
| Missing data | 295 | (100%) | 0 | | 224 | (100%) | 141 | (100%) | 127 | (100%) | 94 | (100%) |
| Resection | | | | | | | | | | | | |
| margin | | | | | | | | | | | | |
| R0 | 0 | | 0 | | 0 | | 0 | | 85 | (66.9%) | 0 | |
| R1 | 0 | | 0 | | 0 | | 0 | | 35 | (27.6%) | 0 | |
| R2 | 0 | | 0 | | 0 | | 0 | | 7 | (5.5%) | 0 | |
| Missing data | 295 | (100%) | 251 | (100%) | 224 | (100%) | 141 | (100%) | 0 | | 94 | (100%) |
| p53 status | | | | | | | | | | | | |
| Mutant | 32 | (10.8%) | 0 | | 0 | | 0 | | 0 | | 0 | |
| Wild-type | 263 | (89.2%) | 0 | | 0 | | 0 | | 0 | | 0 | |
| Missing data | 0 | | 251 | (100%) | 224 | (100%) | 141 | (100%) | 127 | (100%) | 94 | (100%) |
| MSI/MSS | | | | | | | | | | | | |
| status | | | | | | | | | | | | |
| MSI | 0 | | 0 | | 0 | | 2 | (1.4%) | 11 | (8.7%) | 0 | |
| MSS | 0 | | 0 | | 0 | | 136 | (96.5%) | 116 | (91.3%) | 0 | |
| Missing data | 295 | (100%) | 251 | (100%) | 224 | (100%) | 3 | (2.1%) | 0 | | 94 | (100%) |
| Neural | | | | | | | | | | | | |
| invasion | | | | | | | | | | | | |
| Yes | 0 | | 0 | | 0 | | 0 | | 9 | (7.1%) | 0 | |
| No | 0 | | 0 | | 0 | | 0 | | 22 | (17.3%) | 0 | |
| Missing data | 295 | (100%) | 251 | (100%) | 224 | (100%) | 141 | (100%) | 96 | (75.6%) | 94 | (100%) |
| Vascular | | | | | | | | | | | | |
| invasion | | | | | | | | | | | | |
| Yes | 0 | | 0 | | 0 | | 0 | | 10 | (7.9%) | 0 | |
| No | 0 | | 0 | | 0 | | 0 | | 41 | (32.3%) | 0 | |
| Missing data | 295 | (100%) | 251 | (100%) | 224 | (100%) | 141 | (100%) | 76 | (59.8%) | 94 | (100%) |
| Smoking | | | | | | | | | | | | |
| history | | | | | | | | | | | | |
| Current | 0 | | 131 | (52.2%) | 92 | (41.1%) | 0 | | 0 | | 0 | |
| smoker | | | | | | | | | | | | |
| Non-current | 0 | | 120 | (47.8%) | 18 | (8%) | 0 | | 0 | | 0 | |
| smoker | | | | | | | | | | | | |
| Missing data | 295 | (100%) | 0 | | 114 | (50.9%) | 141 | (100%) | 127 | (100%) | 94 | (100%) |
| Adjuvant | | | | | | | | | | | | |
| treatment | | | | | | | | | | | | |
| Yes | 0 | | 0 | | 2 | (0.9%) | 0 | | 0 | | 0 | |
| No | 0 | | 251 | (100%) | 0 | | 0 | | 0 | | 0 | |
| Missing data | 295 | (100%) | 0 | | 222 | (99.1%) | 141 | (100%) | 127 | (100%) | 94 | (100%) |
| Neoadjuvant | | | | | | | | | | | | |
| treatment | | | | | | | | | | | | |

| | | | | | | | | | | | |
|---|---|---|---|---|---|---|---|---|---|---|---|
| Yes | 270 | (91.5%) | 102 | (40.6%) | 69 | (30.8%) | 70 | (49.6%) | 10 | (7.9%) | 0 |

| | | | | | | | | | | |
|---|---|---|---|---|---|---|---|---|---|---|
| No | 25 | (8.5%) | 129 | (51.4%) | 0 | 1 | (0.7%) | 117 | (92.1%) | 0 |

| | | | | | | | | | | |
|---|---|---|---|---|---|---|---|---|---|---|
| Missing data | 0 | 20 | (8%) | 115 | (69.2%) | 70 | (49.6%) | 0 | 94 | (100%) |

^{a}Median survival times were calculated using the Kaplan-Meier method
^{b}Mean survival times were estimated when median survival times cannot be calculated from the data

### Multiplex Immunofluorescence Staining

For the multiplexed immunofluorescence staining, 4 μn thick TMA sections were de-paraffinized, rehydrated and rinsed in distilled H₂O. Two staining protocols were established for the two panels of antibodies: the lymphocyte panel, with CD4, CD8, CD20, FoxP3, CD45RO, and pan-cytokeratin (CK), and the NK/macrophage panel encompassing CD56, NKp46, CD3, CD68, CD163, and pan-CK. The staining procedure was performed as described before (Mezheyeuski et al., 2018). Detailed staining conditions and reagent references are provided in Table 12.

TABLE 12
List of antibodies and dilutions and amplification
reagents used for the multiplex fluorescent IHC.

| | | Antigen | | | | | Amplification/ | |
|---|---|---|---|---|---|---|---|---|
| | | retrieval | | | Host | | enzyme | |
| Panel | Order | * | Marker | Clone | Species | Dilution | reagent^{†} | Company |
| #1 | 1. | pH 9 | CD8a | C8/144B | Mouse | 1:250 | ImPress | Thermo |
| | | | | | | | | Fisher |
| | 2. | pH 9 | CD4 | 4B12 | Mouse | 1:100 | ImPress | Agilent |
| | 3. | pH 6 | CD20 | L26 | Mouse | 1:1500 | Opal HRP | Agilent |
| | 4. | pH 6 | FoxP3 | D6O8R | Rabbit | 1:300 | ImPress | Cell Signaling |
| | 5. | pH 6 | CD45RO | UCHL1 | Mouse | 1:400 | | Thermo |
| | | | | | | | | Fisher |
| | 6. | pH 6 | PanCK | C-11 | Mouse | 1:100 | Opal HRP | Abcam |
| | | | Cytokeratin | AE1/AE3 | Mouse | 1:400 | | Agilent |
| | | | E-cadherin | 36/E | Mouse | 1:2000 | | BD |
| | | | | | | | | Biosciences |
| | 6.^{#} | pH 6 | Melan A | | | 1:100 | ImPress | |
| | 7. | — | DAPI | — | — | — | — | Akoya |
| #2 | 1. | pH 6 | CD3 | F7.2.38 | Mouse | 1:80 | ImPress | Agilent |
| | 2. | pH 6 | CD56 | 123C3 | Mouse | 1:100 | ImPress | Agilent |
| | 3. | pH 6 | NKp46 | NCR1 | Rabbit | 1:150 | ImPress | Thermo |
| | | | | | | | | Fisher |
| | 4. | pH 6 | CD68 | PG-M1 | Mouse | 1:100 | Opal HRP | Agilent |
| | 5 | pH6 | CD163 | 10D6 | Mouse | 1:400 | Opal HRP | Novocastra |
| | 6. | pH 6 | PanCK | C-11 | Mouse | 1:100 | Opal HRP | Abcam |
| | | | Cytokeratin | AE1/AE3 | Mouse | 1:500 | | Agilent |
| | | | E-cadherin | 36/E | Mouse | 1:2000 | | BD |
| | | | | | | | | Biosciences |
| | 6.^{#} | pH 6 | Melan A | | | 1:100 | ImPress | |
| | 7. | — | DAPI | — | — | — | — | Akoya |

* Antigen retrieval performed in microwave oven at 100° C., 15 min.
^{†}Amplification systems ImmPRESS ® HRP or Opal HRP were used: The ImmPRESS ® HRP Anti-Mouse IgG (Peroxidase) (Cat. No: MP-7402-50) and Anti-Rabbit IgG (Peroxidase) Polymer Detection Kits, made in Horse (Cat No: MP-7401-50) (Vector Laboratories); Opal ™ Polymer anti-Rabbit + anti-Mouse HRP Kit (Cat No: ARH1001EA) (Akoya).
^{#}in melanoma instead of cytokeratin/E-cadherin cocktail, Melan A was used to identify malignant tissue vs stroma.

The relevance of cell markers CD206, CD200R, CD36, CD204, macrophage activation protein (MAF), and CD86 for categorizing a cell population as an M2-like macrophage population, as an alternative to the CD68+, CD163+ population, is investigated using corresponding methods and reagents specific for these cell markers.

### Imaging, Image Analysis, and Thresholding

The stained TMAs were imaged using the Vectra Polaris system (Akoya) in multispectral mode at a resolution of 2 pixels/μm. Each of the images was manually reviewed and curated by a pathologist to exclude artefacts, staining defects and accumulation of immune cells in necrotic areas and intraglandular structures. The vendor-provided machine learning algorithm, implemented in the inForm software, was trained to split tissue into three categories: tumor compartment, stromal compartment, or blank areas. The training was performed for each cohort separately by providing a set of the samples that was manually annotated by pathologists. Cell segmentation was performed using DAPI nuclear staining as described (Mezheyeuski et al., 2018). The perinuclear region at 3 μm (6 pixels) from the nuclear border was considered the cytoplasm area. The cell phenotyping function of the inForm software was used to manually define a representative subset of cells positive to expression of each of the markers and a subset of cells negative to all markers. The intensity of the marker expression in selected cells was used to set the thresholds for marker positivity.

Intensity thresholds for the markers were determined in the R programming environment [R Core Team, 2013] by GeneVia Technologies (Tampere, Finland). The marker-specific thresholds were defined by the distributions of the positive and negative cell intensities for that marker. Marker-specific probability density distributions were estimated by smoothing the intensity values with Gaussian kernel estimation with automatic bandwidth detection using the density function of the R package stats. The intensity thresholds for each marker were established as (1) the mean value of the highest intensity of the negative cells and the lowest intensity of the positive cells, if the intensities of the positive and negative cells did not overlap, or (2) as the intensity value which minimized the overall classification error based on the probability density distributions, if there was overlap. The False Positive Rate, True Positive Rate, False Negative Rate, True Negative Rate, and the overall classification error were calculated for each established threshold, i.e. for each marker, and controlled individually. The thresholds were established separately and independently for each tumor type and were applied to the raw output data of the complete cohorts. Every cell was thus characterized as positive or negative for each marker in the panel. This data was used to classify the cell and define its immune subtype (Table 1). Finally, cell counts were normalized against analyzed tissue area size and used as cell density (units per mm²) in further analyses.

### Signature of Immune Activation and Immunoscore®

The signature of immune activation (SIA) was computed as a ratio of CD8+ cell density to the sum of the densities of CD8+ and M2-like cells, or SIA=(CD8 density)/(CD8 density+M2-like density). The Immunoscore® (IS) was generated as described (Pagès et al., 2018). Each tumor in the CRC TMA cohort was represented by TMA cores derived from the central part and the invasive margin of the tumors. The CD3 and CD8-positive cells were defined in each of the regions, thus resulting in four values per case (i.e. CD3 density in tumor center, CD8 density in tumor center, CD3 density at the invasive margin, CD8 density at the invasive margin). The IS was generated as described by computing a mean of the four. In the other cohorts, the TMA cores were obtained from the bulk tumor region, without separation between central parts and invasive margin. Thus, for these tumors two values per case were obtained (CD3 and CD8-positive cell density) and IS was generated by computing a mean of the two. Further, using the mean percentiles, IS was categorized into 3 groups: Low (mean percentile 0-25%), Intermediate (25-70%) and High (70-100%).

### Mutations and Neoantigens

The median values of the numbers of mutations and neoantigens across 19 solid cancers were obtained from The Cancer Immunome Atlas (TCIA) project (tcia.at/home).

### Statistics

Statistical analyses were performed using R (version 3.5.1) and SPSS V20 (SPSS Inc., Chicago, IL). In radically operated CRC stage I-III patients, recurrence-free survival (RFS) was computed as the time from surgery to the first documented disease progression including local recurrence or distant metastases or death due to any reason, whichever occurred first. Overall survival (OS) was the time from surgery to death due to any reason. To estimate relative hazards in both univariate and multivariable models, a Cox proportional hazards model was used. The predictive accuracies of the models were evaluated by 1000-fold bootstrap resampling and by computing the time-dependent area under the receiver operating characteristic curve (iAUC) for each bootstrap sampling. The relative importance of parameters for the estimation of survival risk was computed using the chi squared proportion (χ²).

### REFERENCES

Bi et al. (2021). Tumor and immune reprogramming during immunotherapy in advanced renal cell carcinoma. Cancer Cell, 39, 649-661.
Brierly et al. (Ed.). (2017). TNM Classification of Malignant Tumours (8 ed.). Wiley-Blackwell. Retrieved from ISBN: 978-1-119-26357-9
Charoentong et al. (2017). Pan-cancer Immunogenomic Analyses Reveal Genotype-Immunophenotype Relationships and Predictors of Response to Checkpoint Blockade. Cell Rep, 18, 248-262.
Durante et al. (2020). Single-cell analysis reveals new evolutionary complexity in uveal melanoma. Nat. Commun., 11, 496.
Galon et al. (2006, September 29). Type, density, and location of immune cells within human colorectal tumors predict clinical outcome. Science, 29(313), 1960-1964.
Gomez-Brouchet et al. (2017). CD163-positive tumor-associated macrophages and CD8-positive cytotoxic lymphocytes are powerful diagnostic markers for the therapeutic stratification of osteosarcoma patients: An immunohistochemical analysis of the biopsies from the French OS2006 phase 3. Oncoimmunology, 6(9), e1331193 1-12.
He et al. (2020). Single-cell transcriptome profiling of an adult human cell atlas of 15 major organs. Genome Biol, 21, 294.
Hemdan et al. (2014). The prognostic value and therapeutic target role of stathmin-1 in urinary bladder cancer. Br J Cancer, 111, 1180-1187.
Hugo et al. (2016). Genomic and Transcriptomic Features of Response to Anti-PD-1 Therapy in Metastatic Melanoma. Cell, 165, 35-44.
Huvila et al. (2018). Combined ASRGL1 and p53 immunohistochemistry as an independent predictor of survival in endometrioid endometrial carcinoma. Gynecol Oncol, 149, 173-180.
Jeremiasen et al. (2020). Tumor-Associated CD68(+), CD163(+), and MARCO(+) Macrophages as Prognostic Biomarkers in Patients With Treatment-Naive Gastroesophageal Adenocarcinoma. Front Oncol, 10, 534761.
Lambrechts et al. (2018). Phenotype molding of stromal cells in the lung tumor microenvironment. Nat. Med., 24, 1277-1289.
Lee et al. (2020). Lineage-dependent gene expression programs influence the immune landscape of colorectal cancer. Nat. Genet., 52, 594-603.
Mezheyeuski et al. (2018). Multispectral imaging for quantitative and compartment-specific immune infiltrates reveals distinct immune profiles that classify lung cancer patients. The Journal of pathology, 244, 421-431.
Micke et al. (2016). The Impact of the Fourth Edition of the WHO Classification of Lung Tumours on Histological Classification of Resected Pulmonary NSCCs. J Thorac Oncol, 11, 862-872.
Nagy et al. (2021). Pancancer survival analysis of cancer hallmark genes. Sci. Rep., 11, 6047.
Nodin et al. (2010). Increased androgen receptor expression in serous carcinoma of the ovary is associated with an improved survival. J Ovarian Res, 3(1), 14.
Pagès et al. (2018, May 26). International validation of the consensus Immunoscore for the classification of colon cancer: a prognostic and accuracy study. Lancet, 391(10135), 2128-2139.
Sade Feldman et al. (2018). Defining T Cell States Associated with Response to Checkpoint Immunotherapy in Melanoma. Cell, 175(e1020), 998-1013.
Stromberg et al. (2009). Selective expression of Syntaxin-7 protein in benign melanocytes and malignant melanoma. J Proteome Res, 8, 1639-1646.

1. An in vitro method for the prediction of response to immunotherapy for, or prognosis of survival time of, a subject diagnosed with a cancer, comprising
Measuring, in a tissue affected by said cancer, a first density D1 of a first cell category consisting of cells positive for CD8 and a second density D2 of a second cell category consisting of cells positive for at least one of the following: C1g, the combination of CD68 and CD163; and the combination of CD68 and C1g,
determining a relation between D1 and D2; and
comparing the determined relation to at least one predetermined reference value predictive of response to immunotherapy, or indicative of a survival time, for said subject;
wherein the second cell category represents M2-like macrophages.

2. The method according to claim 1, wherein the method comprises
obtaining a sample of cancerous tissue from said subject;
Measuring, in said tissue sample, a first density D1 of a first cell category consisting of cells positive for CD8 and a second density D2 of a second cell category consisting of cells positive for at least one of the following: C1g, the combination of CD68 and CD163; and the combination of CD68 and C1g ,
determining a relation between D1 and D2;
comparing the determined relation to at least one predetermined reference value indicative of a survival time for said subject; and
determining a prediction of response to immunotherapy or prognosis of survival time for the subject based on the comparison;
wherein the second cell category represents M2-like macrophages.

3. The method according to claim 1 for the prediction of response to immunotherapy for the subject, wherein the predetermined reference values have been determined by
Measuring, in samples of cancerous tissue from each subject in a cohort of subjects diagnosed with said cancer and with a known response to immunotherapy, a first density D1 of a first cell category consisting of cells positive for CD8 and a second density D2 of a second cell category consisting of cells positive for at least one of the following: C1q, the combination of CD68 and CD163; and the combination of CD68 and C1 q,
determining a relation between D1 and D2;
comparing the determined relation to the known survival times of the subjects to obtain an association between the relation and the survival time; and
determining the reference values so as to be predictive of response to immunotherapy for a subject diagnosed with the cancer.

4. The method according to claim 1 for the prognosis of survival time of the subject, wherein the predetermined reference values have been determined by
Measuring, in samples of cancerous tissue from each subject in a cohort of subjects diagnosed with said cancer and with a known survival time, a first density D1 of a first cell category consisting of cells positive for CD8 and a second density D2 of a second cell category consisting of cells positive at least one of the following: C1q, the combination of CD68 and CD163; and the combination of CD68 and C1q,
determining a relation between D1 and D2;
comparing the determined relation to the known survival times of the subjects to obtain an association between the relation and the survival time; and
determining the reference values so as to be indicative of survival time for a subject diagnosed with the cancer.

5. A method of measuring relative cell densities in a sample of cancer affected tissue comprising the steps of measuring a first density D1 of a first cell category consisting of cells positive for CD8 in the tissue sample, and a second density D2 of a second cell category consisting of cells positive for at least one of the following: C1q, the combination of CD68 and CD163; and the combination of CD68 and C1q;
in the tissue sample, and calculating a relation between D1 and D2, wherein the second cell category represents M2-like macrophages.

6. The method according to claim 1, wherein the second cell category consists of cells positive for both CD68 and CD163.

7. The method according to claim 1, wherein the second cell category consists of cells positive for at least one of C1qA, C1qB, and C1qC, and optionally CD68.

8. The method according to claim 1, wherein
(i) the determination of the relation between D1 and D2 comprises calculating the ratio D1/(D1+D2) or D1/D2, or an inverse thereof, or
(ii) wherein the cancer is selected from colorectal cancer, breast cancer, pancreatoduodenal cancer, bladder cancer, lung cancer, melanoma, and gastroesophageal adenocarcinoma, or wherein the determined relation is combined with at least one clinical risk factor in determining a prognosis of survival time for the subject.

9. (canceled)

10. (canceled)

11. The method according to claim 8, wherein the at least one clinical risk factor is selected from the group consisting of subject's sex, microsatellite instability status, tumor sidedness, T stage, N stage, tumor differentiation.

12. The method according to claim 1,
(i) wherein the measurement of cell densities is performed by analysis of gene expression, or
(ii) wherein the measurement of cell densities is performed by counting cells positive for CD8 and cells positive for at least one of the following: C1q, the combination of CD68 and CD163; and the combination of CD68 and C1q, in an analysed tissue area, and optionally normalizing against the size of the analysed tissue area.

13. (canceled)

14. The method according to claim 12, wherein the analysed tissue area comprises both tumour centre and invasive margin.

15. The method according to claim 12, wherein the counting of cells is facilitated by staining of the tissue with detectable antibodies specific for the CD8, CD68, CD163, C1q, C1qA, C1qB, or C1qC to be detected.

16. An in vitro method for the prediction of response to immunotherapy for, or prognosis of survival time of, a subject diagnosed with a cancer, comprising
a) Measuring, in a tissue affected by said cancer, a first concentration C1 of a first group of molecules selected from the group consisting of CD8 and RNA molecules encoding therefore, and a second concentration C2 of a second group of molecules selected from the group consisting of: C1q and RNA molecules encoding therefore;
b) determining a relation between C1 and C2; and
c) comparing the determined relation to at least one predetermined reference value predictive of response to immunotherapy, or indicative of a survival time, for said subject;
wherein the second group of molecules represents M2-like macrophages.

17. The method according to claim 16, wherein the method comprises
obtaining a sample of cancerous tissue from said subject;
performing steps a)-c) of the method according to claim 16; and
determining a prediction of response to immunotherapy or prognosis of survival time for the subject based on the comparison;
wherein the second cell category represents M2-like macrophages.

18. The method according to claim 16, for the prediction of response to immunotherapy for the subject, wherein the predetermined reference values have been determined by
Measuring, in a tissue affected by said cancer, a first concentration C1 of a first group of molecules selected from the group consisting of CD8 and RNA molecules encoding therefore, and a second concentration C2 of a second group of molecules selected from the group consisting of: C1q and RNA molecules encoding therefore;
determining a relation between C1 and C2; and
comparing the determined relation to the known survival times of the subjects to obtain an association between the relation and the survival time; and
determining the reference values so as to be predictive of response to immunotherapy for a subject diagnosed with the cancer.

19. A method of measuring relative molecule concentrations in a sample of cancer affected tissue comprising the steps of measuring a first concentration C1 of a first group of molecules selected from the group consisting of CD8 and RNA molecules encoding therefore in the tissue sample, and a second concentration C2 of a second group of molecules selected from the group consisting of: C1q and RNA molecules encoding therefore; in the tissue sample, and calculating a relation between C1 and C2.

20. (canceled)

21. The method according to 19,
(i) wherein the second group of molecules consists of at least one of C1qA, C1qB, and C1qC, or RNA molecules encoding therefore, or
(ii) wherein the determination of the relation between C1 and C2 comprises calculating the ratio C1/(C1+C2) or C1/C2, or an inverse thereof, or
(iii) wherein the cancer is selected from colorectal cancer, breast cancer, pancreatoduodenal cancer, bladder cancer, lung cancer, melanoma, and gastroesophageal adenocarcinoma, or
(iv) wherein the determined relation is combined with at least one clinical risk factor in determining a prognosis of survival time for the subject.

22. (canceled)

23. (canceled)

24. (canceled)

25. The method according to claim 21, wherein the at least one clinical risk factor is selected from the group consisting of subject's sex, microsatellite instability status, tumor sidedness, T stage, N stage, tumor differentiation.

26. The method according to claim 16, wherein the measurement of concentration is performed by bulk RNA sequencing.
